(19) United States
(12) Patent Application Publication
(10) Pub. No.: US 2013/0338479 A1
(43) Pub. Date: Dec. 19, 2013

(54) Apparatus And Method For Surgical Instrument With Integral Automated Tissue Classifier

(71) Applicants: Brian William Pogue, Hanover, NH (US); Venkataramanan Krishnaswamy, Lebanon, NH (US); Keith D. Paulsen, Hanover, NH (US); Pilar Beatriz Garcia Allende, Munich (DE); Olga Maria Conde, Santander (ES); José Miguel Lopez-Higuera, Santander (ES)

(72) Inventors: Brian William Pogue, Hanover, NH (US); Venkataramanan Krishnaswamy, Lebanon, NH (US); Keith D. Paulsen, Hanover, NH (US); Pilar Beatriz Garcia Allende, Munich (DE); Olga Maria Conde, Santander (ES); José Miguel Lopez-Higuera, Santander (ES)

(73) Assignees: UNIVERSIDAD DE CANTABRIA, Cantabria (ES); THE TRUSTEES OF DARTMOUTH COLLEGE, Hanover, NH (US)

(21) Appl. No.: 13/140,748

(22) PCT Filed: Dec. 18, 2009
(86) PCT No.: PCT/US09/68718

§ 371 Date: Sep. 5, 2013

(30) Foreign Application Priority Data
Dec. 19, 2008 (US) 61139323

(51) Int. Cl. A61B 5/00; A61B 5/145

(52) U.S. Cl. CPC A61B 5/7264; A61B 5/0068; A61B 5/742; A61B 5/0071; A61B 5/14546; A61B 2576/00
USPC 600/408

(57) ABSTRACT

A method and apparatus is described for optically scanning a field of view, the field of view including at least part of an organ as exposed during surgery, and for identifying and classifying areas of tumor within the field of view. The apparatus obtains a spectrum at each pixel of the field of view, and classifies pixels with a kNN-type or neural network classifier previously trained on samples of tumor and organ classified by a pathologist. Embodiments use statistical parameters extracted from each pixel and neighboring pixels. Results are displayed as a color-encoded map of tissue types to the surgeon. In variations, the apparatus provides light at one or more fluorescence stimulus wavelengths and measures the fluorescence light spectrum emitted from tissue corresponding to each stimulus wavelength. The measured emitted fluorescence light spectra are further used by the classifier to identify tissue types in the field of view.

### CLAIM TO PRIORITY

The present application claims priority to U.S. Provisional Patent Application 61/139,323 filed Dec. 19, 2008 the disclosure of which is incorporated herein by reference.

### GOVERNMENT INTEREST

The work described in the present document has been funded in part by National Institutes of Health grants P01CA80139 and P01CA84203. The work has also received funding from the government of Spain through its Ministry of Science and Technology project numbers TEC 2005-08218-C02-02 and TEC 2007-67987-C02-01. The United States Government therefore has rights in the invention described herein.

### FIELD

The present document relates to the field of automated identification of biological tissue types. In particular, this document describes apparatus for use during surgery that examines optical backscatter characteristics of tissue to determine tissue microstructure, and which then classifies the tissue as tumor or non-tumor tissue. The apparatus is integrated into a surgical microscope intended for use in ensuring adequate tumor removal during surgery.

### BACKGROUND

Many tumors and malignancies are treated, at least in part, by surgical removal of malignant tissue. It is known that patient survival can be reduced if malignant tissue is left in operative sites; so many such operations involve removing considerable adjacent normal tissue along with the tumor to ensure that all possible tumor is removed. It is also true that removal of excessive normal tissue is undesirable as it may cause loss of function, pain and morbidity.

Malignant tumors are often not encapsulated; the boundary between tumor and adjacent normal tissue may be uneven with projections and filaments of tumor extending into the normal tissue. After initial removal of a tumor, it is desirable to inspect boundaries of the surgical cavity to ensure all tumor has been removed; if remaining portions of tumor are detected, additional tissue may be removed to ensure complete tumor removal.

Conventionally, boundaries of the surgical cavity have been inspected visually by a surgeon. A surgical microscope may be used for this inspection, but small projections and filaments of tumor may escape detection because tumor tissue often at least superficially resembles normal tissues of the organ within which the tumor first arose. Further, removed tissue may be sectioned and inspected by a pathologist to ensure that a rim of normal tissue has been removed along with the diseased tissue; this may be done intraoperatively using frozen sections and followed up with microscopic evaluation of stained sections for tumor-specific features—but stained sections are typically not available until days after completion of the surgery. Further, it is generally not practical to examine frozen or stained sections of organ portions remaining in a patient after tumor resection.

Studies of contrast-enhancement technologies other than that described herein have shown an increase in survival and a decrease in morbidity when used to assist a surgeon in identifying remaining tumor tissue in an operative site. For example, use by a surgeon of surface fluorescence microscopy to locate and remove remaining tumor portions labeled with metabolites of 5-aminoleulinic acid (5-ALA) has been shown to enhance survival in malignant glioma patients. It is expected that devices that help a surgeon ensure complete tumor removal while minimizing removal and damage to normal tissue will enhance survival and minimize morbidity in subjects having other tumor types.

It is therefore desirable to assist a surgeon in identifying tumor tissue remaining in operative sites in real time during surgery.

In one embodiment, an instrument for automated identification of tissue types and for providing guidance to a surgeon during surgical procedures includes a multi-wavelength optical system for projecting light from a source onto tissue, to illuminate a confined spot of the tissue. A scanner directs the illuminated spot across the tissue in raster form. A spectrally sensitive detector receives light from the optical system in order to produce measurements at a plurality of wavelengths from the illuminated spot on the tissue. A spectral processing classifier determines a tissue type associated with each of the plurality of pixels of the image, and a display device displays tissue type of the plurality of pixels of the image to the surgeon.

In one embodiment, a method of performing tumor removal from tissue includes illuminating a surgical cavity in the tissue with a beam of light, the beam of light illuminating a spot sufficiently small on the tissue that a majority of scattered light is singly scattered. The scattered light from the tissue is received and measured with a spectrally sensitive detector having a dispersive device and an array of photodetector elements. Measurements from the spectrally sensitive detector are adjusted for hemoglobin in the tissue; and scatter parameters extracted from the measurements. The tissue is classified (at least as tumor tissue and normal organ tissue) according to the scatter parameters, and the tissue classification information is displayed. At least some tissue that is classified as rapidly proliferating is removed.

In one embodiment, a method of mapping tissue types in an exposed organ includes illuminating the tissue with a beam of light, the beam of light being scanned across the tissue, the beam of light illuminating a plurality of spots sufficiently small on the tissue that a majority of scattered light is singly scattered. For each illuminated spot on the tissue, the scattered light from the tissue is received and measured with a spectrophotometer. Measurements from the spectrophotometer are adjusted for hemoglobin in the tissue, and scatter parameters are extracted from the measurements. The tissue is classified according to the scatter parameters, at least as normal organ cells and tumor cells. Tissue classification information for each spot of the plurality of spots is displayed. The classification information for each spot is portrayed as a pixel of an image, the image thereby portraying a map of tissue types identified on the tissue.

### SUMMARY

A method and apparatus is described for optically scanning a field of view, the field of view incorporating at least part of an organ as exposed during surgery, and for identifying and classifying areas of tumor within the field of view. The apparatus obtains a spectrum at each pixel of the field of view, and classification of pixels is performed by a K-Nearest-Neighbor type classifier (kNN-type classifier) previously trained on samples of tumor and organ that have been classified by a pathologist. Embodiments using various statistical and textural parameters extracted from each pixel and neighboring pixels are disclosed. Results are displayed as a color-encoded map of tissue types to the surgeon.

### BRIEF DESCRIPTION OF THE DRAWINGS

FIG. 1 is a block diagram of a system for automatically identifying tumor tissue and for providing guidance to a surgeon during surgery.

FIG. 2 is a block diagram of an alternative embodiment of an imaging head for the system.

FIG. 3 is a flowchart of a method of determining a training database for a kNN-type classifier for identifying tumor tissue.

FIG. 4 is a flowchart of a method of determining types of tissue in a field of view and providing guidance to a surgeon during surgery.

FIG. 5 is a block diagram of an enhanced embodiment of a system for automatically identifying tumor tissue and for providing guidance to a surgeon.

FIG. 6 is a block diagram of an alternative embodiment of the handheld scan head of the embodiment of FIG. 5, wherein a circular mirror is used in place of the annular mirror of FIG. 5.

### DETAILED DESCRIPTION OF THE EMBODIMENTS

Localized reflectance measurements of tissue are dependent on local microstructure of the tissue. Since microstructure of tumor tissue often differs in some ways from that of normal tissue in the same organ, localized reflectance measurement of tumor tissue may produce reflectance readings that differ from those obtained from localized reflectance measurements of normal tissue in the same organ.

In a study, reflectance spectrographic measurements of necrotic tumor tissue were shown to vary as much as 50% from measurements of normal tissue, and spectroscopic reflectance measurements of rapidly dividing malignant tumor tissue were shown to vary by as much as 25% from measurements of normal tissue of the type from which the tumor tissue arose.

Most normal organs have at least some degree of heterogeneity, often including such structures as ducts and vessels as well as organ stroma, and organs may be in close proximity to other structures such as nerves. The normal organ stroma of many organs, including kidneys, adrenals, and brains, also varies from one part of the organ to another. The net effect is that there are often multiple normal tissue types in an organ.

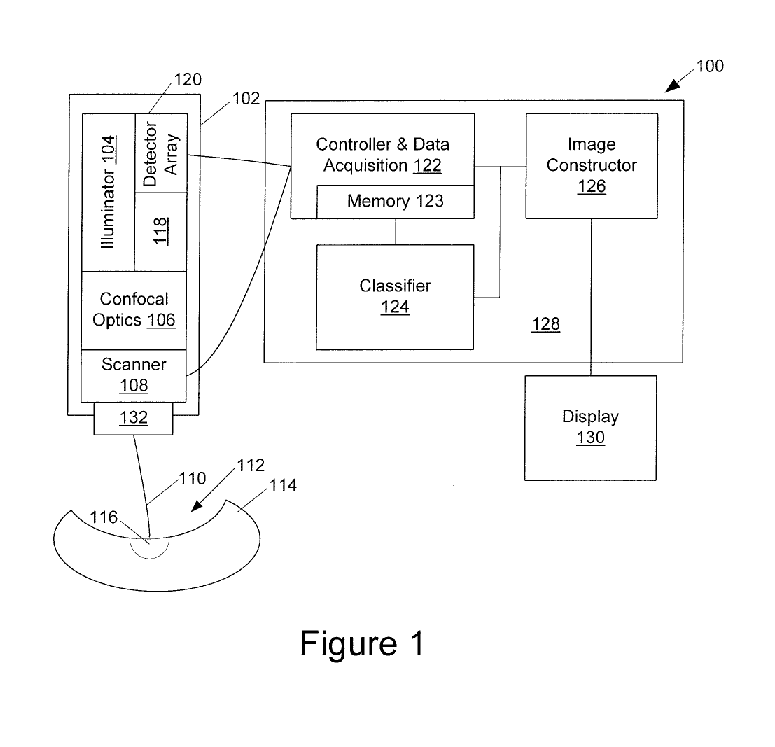

An instrument 100 for assisting a surgeon in surgery is illustrated in FIG. 1. The instrument has an imaging head 102 that is adapted for being positioned over an operative site during surgery. Imaging head has an illuminator subsystem 104 that provides a beam of light through confocal optics 106 to scanner 108. Scanner 108 scans the beam of light 110 through objective lens system 132 onto an operative cavity 112 in an organ 114. A tumor portion 116 may be present in a field of view over which scanner 108 directs beam 110 in cavity 112 in organ 114. Light scattered from the organ 114 and tumor 116 is received through scanner 108 and confocal optics 106 into a spectral separator 118 into a photodetector array 120. Spectral separator 118 is typically selected from a prism or a diffraction grating, and photodetector array 120 is typically selected from a charge-coupled-device (CCD), or CMOS sensor having an array of detector elements, or may be multiple photomultiplier tubes or other photodetector elements as known in the art of photosensors.

Signals from photodetector array 120 incorporate a spectrum of received scattered light for each spot illuminated as scanner 108 raster-scans a field of view on organ 114 and tumor 116, and are passed to a controller and data acquisition subsystem 122 for digitization and parameterization; scanner 108 operates under direction of and is synchronized to controller and data acquisition subsystem 122.

Digitized and parameterized signals from photodetector array 120 are passed to a classifier 124 that determines a tissue type of tissue for each location illuminated by beam 110 in organ 114 or tumor 116, and an image is constructed by image constructor and recorder 126. In an embodiment, conventional optical images of the operative site and images of maps of determined tissue types are constructed. Controller and data acquisition subsystem 122, classifier 124, and image constructor 126 collectively form an image processing system 128, which may incorporate one or more processors and memory subsystems. Constructed images, including both conventional optical images and maps of tissue types are displayed on a display device 130 for viewing by a surgeon.

In an alternative embodiment, a diverter or beam-splitter (not shown in FIG. 1) as known in the art of surgical microscopes, may be provided to permit direct viewing by a surgeon through eyepieces (not shown). In an alternative embodiment, digitization may be performed at detector array 120 instead of controller and data acquisition system 122.

In a particular embodiment, illuminator 104 is a tungsten halogen white light source remotely located from imaging head 102, but coupled through an optical fiber into imaging head 102. In this embodiment, the beam 110 illuminates a spot of less than one hundred microns diameter on the surface of tumor 116 and organ 114 and contains wavelengths ranging from four hundred fifty to eight hundred nanometers. The spot size of less than one hundred microns diameter was chosen to avoid excessive contributions to the received light from multiple scatter in organ 114 and tumor 116 tissue; with small spot sizes of under one hundred microns diameter a majority of received light is singly scattered.

In this embodiment, confocal optics 106 incorporates a beamsplitter for separating incident light of the beam from light, hereinafter received light, scattered and reflected by organ 114 and tumor 116. The received light is focused on a one hundred micron diameter optical fiber to serve as a detection pinhole, and light propagated through the fiber is spectrally separated by a diffraction grating and received by a CCD photodetector to provide a digitized spectrum of the received light for each scanned spot.

The optical system, including confocal optics 106, scanner 108, and objective 132 has a depth of focus such that the effective field of view in organ 114 and tumor 116 is limited to a few hundred microns.

Scanner 108 may be a galvanometer scanner or a rotating mirror scanner as known in the art of scanning optics. Scanner 108 moves the spot illuminated by beam 110 over an entire region of interest of organ 114 and tumor 116 to form a scanned image. Spectra from many spot locations scanned on the surface of organ 114 and tumor 116 in a field of view are stored in a memory 123 as pixel spectra of an image.

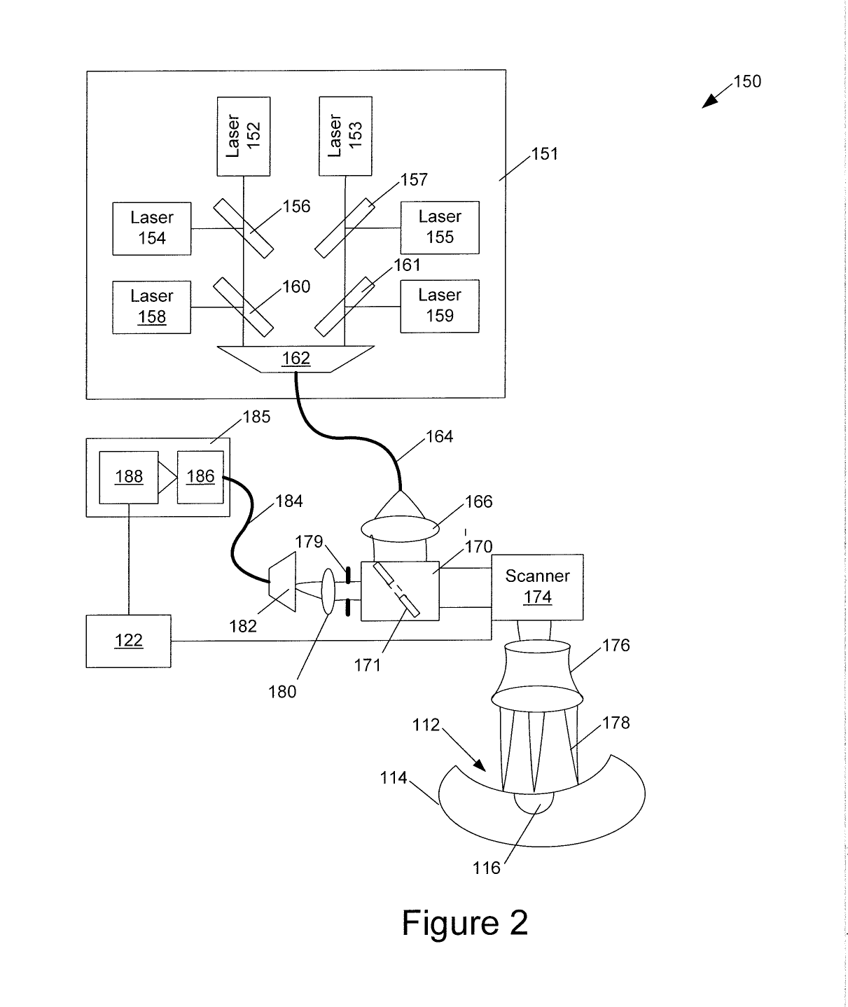

FIG. 2 illustrates an alternative head embodiment 150. As shown, an illuminator 151 has several lasers. In a particular embodiment there are six lasers 152, 153, 154, 155, 158, and 159. Each laser operates at a different wavelength; in this particular embodiment wavelengths of 405, 473, 532, 643, 660, and 690 nanometers are used. In variations of this embodiment, additional lasers at other or additional wavelengths are used. Beams from lasers 152, 153, 154, 155, 158, and 159 are combined by dichroic mirrors 156, 157, 160, 161 and combined and coupled into an optical fiber 164 by coupler 162. Light from illuminator 151 is therefore composite light from a plurality of monochromatic laser light sources.

Light from illuminator 151 is directed by lens 166 into separator 170 containing a mirror 171. Light from illuminator 151 leaves separator 170 as an annular ring and is scanned by scanner 174. Scanner 174 may incorporate a rotating mirror scanner, an X-Y galvanometer, a combination of a rotating mirror in one axis and galvanometer in a second axis, or a mirror independently steerable in two axes.

Light from scanner 174 is directed through lens 176 onto the organ 114 and tumor 116 in operative cavity 112. Light, such as light 178 scattered by the organ 114 and tumor 116 is collected through lens 176 and scanner 174 into separator 170 in the center of the annular illumination. In this embodiment, lens 176 is a telecentric, color-corrected, f-theta scan lens, in one particular embodiment this lens has a focal length of approximately eight centimeters, and is capable of scanning a two by two centimeter field. Light in the center of the beam is passed by separator 170 through an aperture 179, a lens 180 and a coupler 182 into a second optical fiber 184. Aperture 179 may be an effective aperture formed by one or more components of separator 170 or may be a separate component.

Optical fiber 184 directs the light into a spectrally sensitive detector 185, or spectrophotometer, having a dispersive device 186, such as a prism or diffraction grating, and a photosensor array 188. Photosensor array 188 may incorporate an array of charge coupled device (CCD) photodetector elements, complimentary metal oxide semiconductor (CMOS) photodetector elements, P-Intrinsic-N (PIN) diode photodetector elements, or other photodetector elements as known in the art of photosensors. Signals from photosensor array 188 enter the controller and data acquisition system 122 of image processing system 128 (FIG. 1), and scanner 174 operates under control of controller and data acquisition system 122. Remaining elements of image processing system 128, as well as display 130 are similar to those of FIG. 1 and will not be separately described here.

In the embodiment of FIG. 2, illumination light from annular mirror 171 forms a hollow cone, and received light is received from within the center of the illumination cone. This arrangement helps to reject light from specular reflection at surfaces of the organ 114 and tumor 116. This arrangement may be achieved by using a ring-shaped mirror 171 in separator 170, or in another variation by swapping the illumination entrance and spectrometer exit ports of separator 170 and using a small discoidal mirror in separator 170.

In an alternative embodiment, similar to that of FIG. 2, lasers having wavelengths from six hundred to nine hundred nanometers are used.

Once digitized, the pixel spectra are corrected for spectral response of the instrument 100. The corrected spectra are parameterized for hemoglobin concentration and degree of oxygenation by curve-fitting to known spectra of oxygenated HbO and deoxygenated Hb hemoglobin. The spectra are also parameterized for received brightness in the six hundred ten to seven hundred eighty five nanometer portion of the spectrum, which is a group of wavelengths where hemoglobin absorption is of less significance than at shorter wavelengths. The Hb and HbO parameters are used for correction of the scatter parameters.

The scattered reflectance and average scattered power at each of several wavelengths in the obtained spectra are calculated using the empirical equation:

I_{R}=Aλ^{-b}exp(−kc(d(HbO(λ))+(1−d)Hb(λ)))

where λ is wavelength, A is the scattered amplitude, b is the scattering power, c is proportional to the concentration of whole blood, k is the path length of incident light in the organ 114 and tumor 116 tissue, and d is the hemoglobin oxygen saturation fraction. In the embodiment of FIG. 2, the wavelengths of each laser are used in the equation. An average scattered reflectance I_{RAVG} is determined by integrating I_{R} over the wavelength range from the six hundred ten to seven hundred eighty five nanometers to provide an average reflectance.

The extracted reflectance and scatter power, and average scatter parameters are then unity normalized according to the mean of all parameters of the same type throughout the scanned image, and dynamic range compensation is performed, before these parameters are used by classifier 124.

There are many different organs found in a typical human body. Each organ has one or several normal tissue types that have scatter parameters that in some cases may differ considerably from scatter parameters of normal tissue types of a different organ. Further, abnormal tissue, including tissue of a tumor, in one organ may resemble normal tissue of a different organ—for example a teratoma on an ovary may contain tissue that resembles teeth, bone, or hair. Metastatic tumors are particularly likely to resemble tissue of a different organ. For this reason, in an embodiment the classifier is a K-Nearest Neighbors (kNN) classifier 124 that is trained with a separate training database for each different organ type that may be of interest in expected surgical patients. For example, there may be separate training databases for prostates containing scatter information and classification information for normal prostate tissues and prostate tumors, another for breast containing scatter information for normal breast and breast tumors, another for pancreas containing scatter information for normal pancreatic tissues and pancreatic tumors, and another for brain containing scatter information for normal brain tissues as well as brain tumors including gliomas.

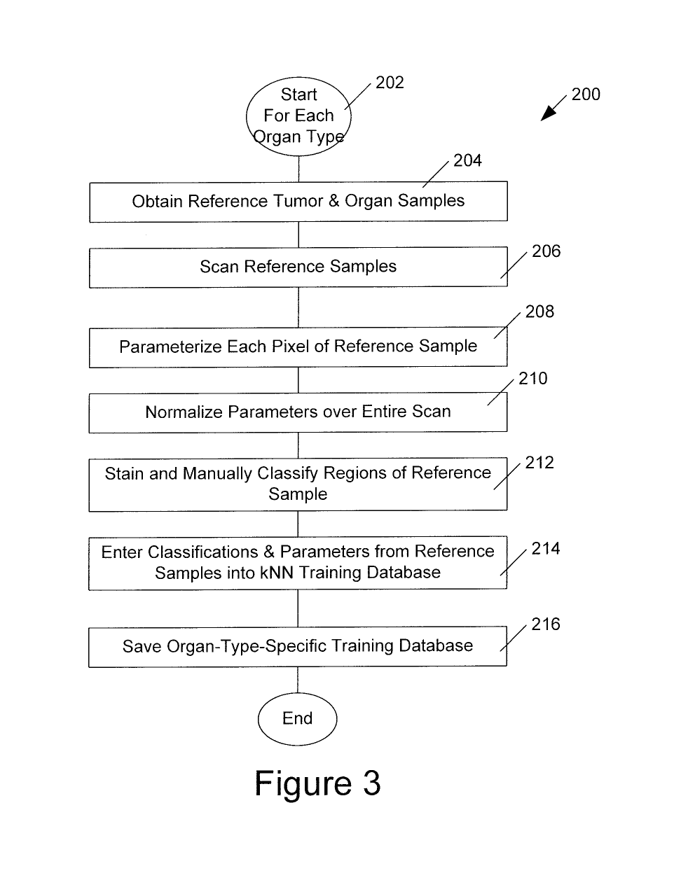

The kNN classifier 124 is therefore trained according to the procedure 200 illustrated in FIG. 3 for each organ type of interest in a group of expected surgical patients. Samples of organs with tumors of tumor types similar to those of expected surgical patients are obtained 204 as reference samples. The reference samples are scanned 206 with an optical system 102 like that previously discussed with reference to FIG. 1 to generate pixels of a reference image. The reference image is parameterized 208 and normalized 210 in the same manner as pixels of images to be obtained during surgery and as discussed above. The reference samples are then fixed and paraffin encapsulated. A surface slice of each sample is stained with hematoxylin and eosin as known in the art of Pathology, and subjected to inspection by one or more pathologists. The pathologists identify particular regions of interest according to tissue types seen in the samples 212. The tissue is classified according to tissue types of interest during cancer surgery, including normal organ capsule and stroma, necrotic tumor tissue, rapidly dividing tumor tissue, fibrotic regions, vessels, and other tissue types that are selected according to the tumor type and organ type.

The parameters for pixels in regions of interest 214 are entered with the pathologist's classification for the region into the training database for the kNN classifier 124. After the reference samples for organs of this type are processed, an organ-specific database is saved 216 for use in surgery.

In a study, similar hardware having a mechanical scanning arrangement instead of a mirror scanner but capable of determining the same reflectance, Hb, and HbO2 parameters, was used to scan samples of pancreatic and prostate tumors grown in rodents. Once scanned to determine a training parameter set corresponding to in-vivo tissue parameters, a surface slice of each sample was encapsulated, fixed, stained with hematoxylin and eosin as known in the art of Pathology, and subjected to inspection by a pathologist. The pathologist identified particular regions of interest in the sections according to tissue types seen in the sections. These included:
epithelial cells with low nucleus to cytoplasm ratio (these are believed to be mature tumor cells);
epithelial cells with high nucleus to cytoplasm ratio (these are believed to be proliferating tumor cells);
fibrotic regions of early fibrosis;
fibrotic regions of intermediate fibrosis;
fibrotic regions of mature fibrosis;
regions of exudative necrosis; and
regions of focal necrosis.
   It should be noted that the tumor type being classified in this experiment was a tumor of an epithelial cell type. The parameters for a subset of pixels of each region of interest, together with the pathologist's classifications were used to train a kNN (k-Nearest-Neighbors) classifier.

Performance of the kNN classifier against unknown pixel data was verified by classifying a different subset of pixels of the same regions with the kNN classifier with a high degree of consistency.

The kNN classifier 124 operates by finding a distance D between a sample set of parameters s corresponding to a particular pixel P and parameter sets in its training database. For example, in an embodiment, at each particular pixel P, if there are M entries in the training database, M distances are calculated from measurements according to the formula

D(pₛ,pₙ)=\sqrt{(Aₛ−Aₙ)²+(bₛ−bₙ)²+(I_{avgs}−I_{avgn})²} for n=1 to M.

The scanned pixel P is classified according to the classification assigned in the training database to parameter sets giving the smallest distance D. In alternative embodiments, distance D is computed using other statistical distances instead of the formula above, such as those given by Mahalanobis, Bhattacharyya, or other distance formulas as known in the art of statistics. It is expected that a kNN classifier using the Mahalanobis distance formula may provide more accurate classification than the Euclidean distance formula.

In a particular embodiment, each pixel spectra is obtained by measuring intensity at six discrete wavelengths in the 400-700 nanometer range. In alternative embodiments, additional wavelengths are used.

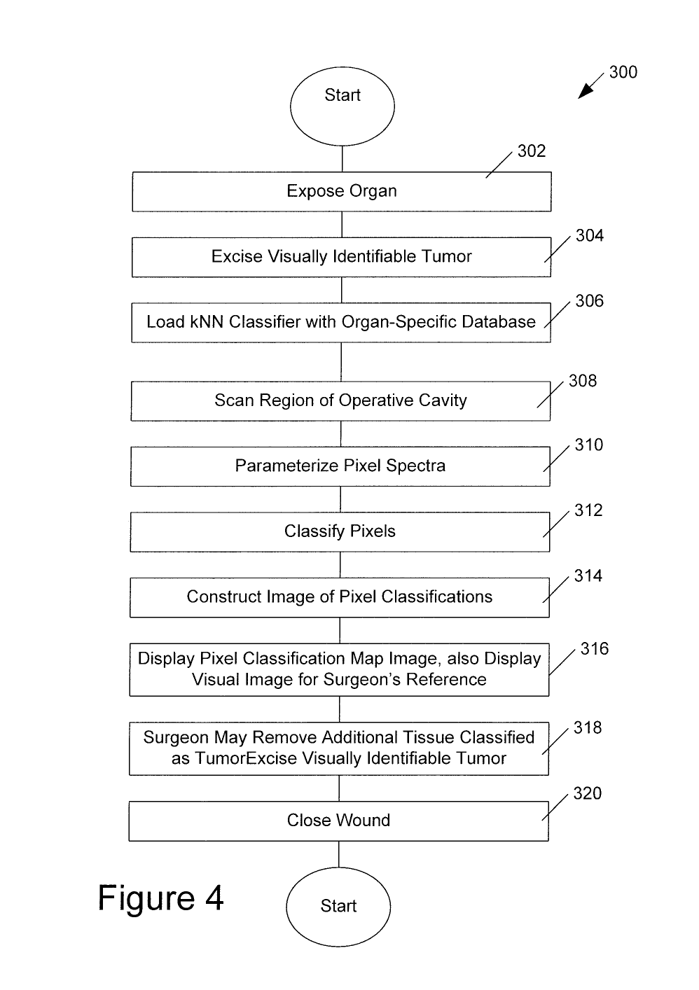

In the surgical procedure 300 illustrated in FIG. 4, the organ of interest is exposed 302 by the surgeon. The surgeon then excises 304 those portions of tumor that are visually identifiable as such as known in the art of surgery. Meanwhile, the kNN classifier 124 is loaded 306 with an appropriate organ-specific database saved at the end of the reference classification procedure of FIG. 3.

A region of interest in the operative cavity is scanned 308 by optical system 102, an array of pixel spectra obtained is parameterized 310, the pixels are classified 312 by classifier 124, and a map image of the classifications is constructed 314. The classifier classifies the tissue at least as tumor tissue and normal organ tissue, in an alternative embodiment the classifier classifies the tissue as normal organ tissue, rapidly proliferating tumor tissue, mature tumor tissue, fibrotic tissue, and necrotic tissue. In an embodiment, the map image is color encoded pink for mature tumor tissue, red for rapidly proliferating tumor tissue, and blue for normal organ tissue. In alternative embodiments, other color schemes may be used. The classification map is displayed 316 to the surgeon. The surgeon may also view a corresponding raw visual image to orient the map in the region of interest. The surgeon may then excise 318 additional tumor, and repeat steps 308-318 as needed before closing 320 the wound.

In an alternative embodiment, in addition to the three scatter-related parameters heretofore discussed with reference to kNN classifier 124, additional parameters are defined for each pixel both during training of the classifier and intraoperatively. These additional parameters include statistics such as mean, standard deviation, a skew measure, and a kurtosis measure, and in alternative embodiments include additional parameters derived from texture features such as contrast, energy, entropy, correlation, sum average, sum entropy, difference average, difference entropy and homogeneity, of reflectance in a window centered upon the pixel being classified. These parameters are collectively referred to as statistical parameters. Adding these parameters to the parameters used for classification by the kNN classifier 124 appears to improve accuracy of the resulting map of tissue classifications. In this classifier, an alternative formula, having weights for each parameter, for calculating distance was used, according to the Bhattacharya statistical distance. In this measure, the difference in a scattering parameter p, with p=1,2, . . . , 15, between two tissue subtypes, i and j, is given by:

$${J}_{ij}^{p} = \frac{1}{4} {{( {μ}_{j} - {μ}_{i} )}^{T} [ {\sum }_{i} + {\sum }_{j} ]}^{- 1} ( {μ}_{j} - {μ}_{i} ) + \frac{1}{2} ln ( \frac{\vert {\sum }_{i} + {\sum }_{j} \vert}{2 {( \vert {\sum }_{i} \vert \cdot \vert {\sum }_{j} \vert )}^{\frac{1}{2}}} )$$

where μᵢ and Σᵢ are the mean and the variance matrix of p for tissue sub-type i. Further, Jᵢⱼ is the distance between sub-types i and j. For smaller window sizes, which means that mostly vicinity regions will be within the same tissue sub-type, the mean scattering power is always selected as the most discriminating feature.

In this embodiment, experiments have been performed using window sizes of from four by four pixels to twelve by twelve pixels centered upon the pixel being classified. This classifier gave classifications that more closely matched those given by the pathologist than those provided by using only scatter parameters in the classifier.

In an alternative embodiment 400 having enhanced capabilities, a different light source 401 is used which differs from the light source 151 illustrated in the embodiments of FIG. 2. Light source 401 has a broad spectrum, or white-light-producing element that provides radiation across a wide selection of wavelengths ranging from the visible through the infrared. In an embodiment, the light producing element is a supercontinuum laser 402 having significant output ranging from wavelengths of nearly four hundred nanometers to greater than two thousand nanometers. Supercontinuum lasers covering this broad spectral range are available from NKT Photonics, Birkerod, Denmark, although other sources may be used.

Light from laser 402 is passed through a filter 404 that passes a wavelength range of particular interest for determining scatter signatures of normal and tumor cells, while blocking light at the infrared end of the spectrum that may cause undue heating of components and requires detectors made of exotic materials other than silicon. In an embodiment, filter 404 passes a range of radiation from 400 to 750 nanometers, in an alternative embodiment laser 402 emits light of wavelengths 400 nanometers and longer, while filter 404 is a high-pass filter that passes wavelengths shorter than 750 nanometers.

Light passed by bandpass filter 404 is divided into two beams by a beamsplitter 406. One beam from beamsplitter 406 passes to a high speed, electronically operated, optical beam switching device 410. A second beam from beamsplitter 406 passes through a tunable filter 408 and then to switching device 410. In an embodiment, tunable filter 408 is an acousto-optic tunable filter; in an alternative embodiment tunable filter 408 is a rotary filter having several bandpass elements having different center frequencies and which rotates under computer control to change wavelengths of light passing through filter 408. An alternative embodiment filter 408 is a liquid crystal tunable.

Computer-controlled optical switch 410 selects light from a desired path from tunable filter 408 or beamsplitter 406, and passes the light to a fiber coupler 412. Fiber coupler 412 couples the light into a source optical fiber 414. In an embodiment, optical fiber 414 is a single mode fiber of about five microns diameter. The entire light source 401 operates under control of a local microcontroller 416.

As with the embodiment of FIG. 2, light from optical fiber 414 passes through a lens 420 into separator 422 containing an annular mirror 424. Light from fiber 414 leaves separator 422 as an annular ring and is scanned by scanner 428. Scanner 428 may incorporate a rotating mirror scanner, an X-Y galvanometer, a combination of a rotating mirror in one axis and galvanometer in a second axis, or a mirror independently steerable in two axes.

Light from scanner 428 is directed through lens 430 onto the organ 114 and tumor 116 tissues in operative cavity 112. The scanner 428 causes the light to scan across an opening or window of handheld probe 426 beneath lens 430, this light is illustrated at several scanned beam 432 positions. Light, such as light 432 scattered by the organ 114 and tumor 116 tissues is collected through the same lens 430 and scanner 428 into separator 422, where it passes through an aperture 423. At least some of light 432 is returned to separator 422 in the center of the beam, and passes through another lens 440 and coupler 444 into a receive fiber 442.

In an embodiment, lens 430 is a telecentric, color-corrected, f-theta scan lens, in one particular embodiment this lens has a focal length of eight centimeters, and is capable of scanning a two by two centimeter field. In an embodiment, aperture 423 may be an effective aperture formed by one or more components of separator 422, such as a central hole in mirror 424, or may be a separate component.

Optical fiber 422 directs the light into a spectrally sensitive detector 448, or spectrophotometer, having a dispersive device 450, such as a prism or diffraction grating, and a photosensor array 452. Photosensor array 452 may incorporate an array of charge coupled device (CCD) photodetector elements, complimentary metal oxide semiconductor (CMOS) photodetector elements, P-Intrinsic-N (PIN) diode photodetector elements, or other photodetector elements as known in the art of visible and near-infrared-sensitive photosensors. Signals from photosensor array 452 enter the controller and data acquisition system 460 of image processing system 462. Scanner 428, as well as light source 401 through its microcontroller 416 operates under control of controller and data acquisition system 460. Remaining elements of image processing system 462, as well as display 464, are similar to those of image processing system 128 and display 130 of FIG. 1 and will not be separately described here.

In a scattering-based mode of operation, beam switch 410 passes light from filter 404 into fiber coupler 412, and thence to tumor 116. Photosensor array 452 receives and performs spectral analysis of light scattered by tissue of organ 114 and tumor 116, and received through spectrally sensitive detector 448, and processing system 462 uses a kNN classifier as previously discussed to classify tissue as tumor tissue or normal tissue. In an alternative embodiment, the processing system may use another classifying scheme known in the art of computing such as artificial neural networks, and genetic algorithms.

In particular alternative embodiments, the processing system uses an Artificial Neural Network classifier, in another embodiment a Support Vector Machine classifier, in another a Linear Discriminant Analysis classifier, and in another a Spectral Angle Mapper classifier; all as known in the art of computing.

In a fluorescence-based mode of operation, the subject within which organ 114 and tumor 116 tissue lies is administered a fluorescent dye containing either a fluorophore or a prodrug such as 5-aminolevulinic acid (5-ALA) that is metabolized into a fluorophore such as protoporphyrin-IX. Fluorescent dyes may also include a fluorophore-labeled antibody having specific affinity to the tumor 116. With both administered fluorophore or prodrug dyes, fluorophore concentrates in tumor 116 to a greater extent than in normal organ 114. In alternative fluorescence operation, one or the other, or both, of organ 114 and tumor 116 may contain varying concentrations of endogenous fluorophores such as but not limited to naturally occurring protoporphyrin-IX or beta-carotene.

In the fluorescence-based mode of operation, beam switch 410 passes light from tunable filter 408 into fiber coupler 412, and thus into fiber 414 and handheld probe 426. In this mode, tunable filter 408 is configured to pass light of a suitable wavelength for stimulating fluorescence by the fluorophore in organ 114 and tumor 116, while significantly attenuating light at wavelengths of fluorescent light emitted by the fluorophore. Although detector 448 is spectrally sensitive, attenuation of light at wavelengths of fluorescent light by filter 408 increases sensitivity and reduces susceptibility of the system to dirt in the optical paths.

Fluorescent light emitted by fluorophore in organ 114 and tumor 116 is received through lens 430, scanner 428, separator 422, lens 440, coupler 444, fiber 446, into spectrally sensitive detector 448. Spectrally sensitive detector 448 detects the light and passes signals representative of fluorescent light intensity at each pixel of an image of the tissue scanned by scanner 428 as a fluorescence image into image processor 462.

The tunable filter 408 is thereupon changed to other wavelengths and the three specular scatter parameters are determined as discussed above. Image processor 462 thereupon uses the fluorescence intensity and spectrum information as additional information with the three spectral parameters discussed above to classify tissue types in tissue, and displays the tissue classification information to the surgeon. The fluorescence spectrum information is used during classification to allow spectral unmixing of drug and prodrug fluorescence from fluorescence from endogenous fluorophores in tissue. After unmixing, bulk fluorescence is calculated for the given excitation wavelength. Image processor 462 may also present an image of fluorescence to the surgeon.

In an embodiment the ratio of fluorescence intensity to scattered irradiance at the excitation wavelength, which is collected as a part of the scatter mode data, is used as a normalized fluorescence value by the classifier.

In an embodiment, the ratio of fluorescence intensity to scattered irradiance is computed for several different stimulus wavelengths and several different fluorescence wavelengths; in this embodiment these additional ratios are used by the classifier to better distinguish different fluorophores in tumor 116 and organ 114 tissues, and thus to provide improved classification accuracy.

In a fluorescence-only mode of operation of embodiment, fluorescence mode information is used by the classifier without the scattering parameters discussed above; in a synergistic mode of operation both fluorescence and scattering parameters are used by the classifier at each pixel to provide enhanced tissue classification information.

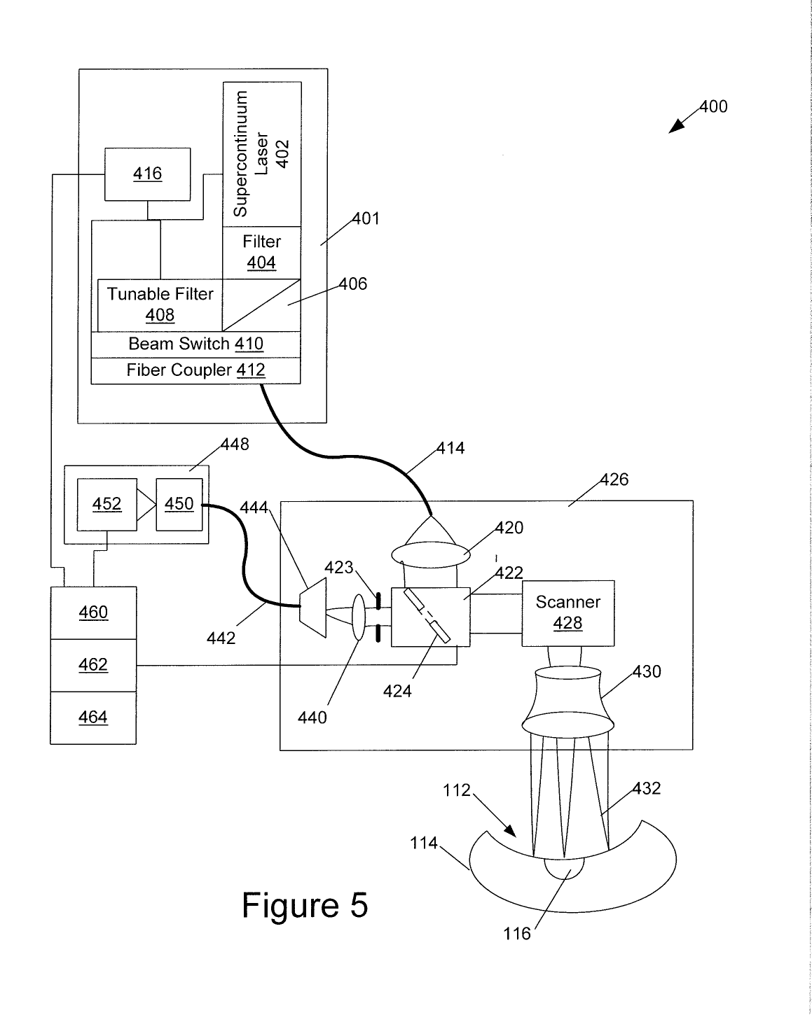

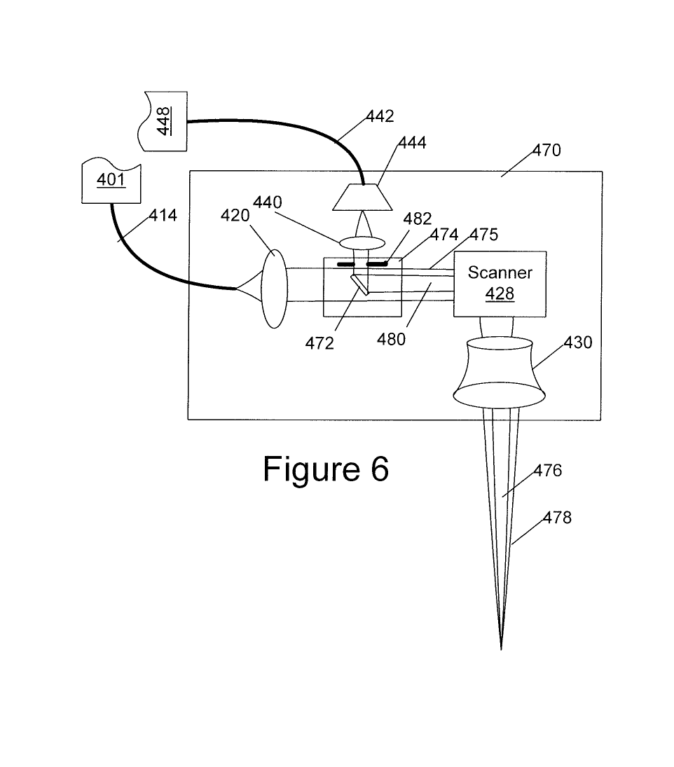

In an alternative embodiment, as illustrated in FIG. 6, resembling that of FIG. 5, a light source 401 identical to that previously discussed with reference to FIG. 5 is used, driving a source optical fiber 414. Similarly, receive optical fiber 442 couples to a spectrally sensitive detector 448 like that previously discussed with reference to FIG. 5. As with FIG. 5, detector 448 feeds an image processing system as previously discussed, in the interest of brevity discussion of the light source, spectrally sensitive detector, and image processing system will not be repeated here.

The embodiment of FIG. 6 differs from the embodiment of FIG. 5 in that handheld probe 470 uses a modified separator 474 having a discoidal mirror 472 instead of the annular mirror 424 of separator 422 of probe 426 of FIG. 5. Source fiber 414 projects light from source 401 through lens 420 around discoidal mirror 472 to form an annular source beam that leaves separator 474 and enters scanner 428; as previously discussed scanner 428 scans this annular illumination 475 through telecentric lens 430 across organ and tumor. Scattered light is received through lens 430 in a central portion 476 of scanned beam 478, and into separator 474 as a received beam 480 contained within annular illumination 475. Discoidal mirror 472 reflects received beam 480 through an aperture 482, which is focused by lens 440 into receive coupler 444 and receive fiber 442 for transmission to the detector.

While the invention has been particularly shown and described with reference to a preferred embodiment thereof, it will be understood by those skilled in the art that various other changes in the form and details may be made without departing from the spirit and scope of the invention. It is to be understood that various changes may be made in adapting the invention to different embodiments without departing from the broader inventive concepts disclosed herein and comprehended by the claims that follow.

What is claimed is:

1. An instrument for automated identification of tissue types and for providing guidance to a surgeon during surgical procedures comprising:
a multi-wavelength optical system for projecting light from a source onto tissue to illuminate a confined spot of the tissue;
a scanner for directing the illuminated spot across the tissue in raster form;
a spectrally sensitive detector for receiving light from the optical system in order to produce measurements at a plurality of wavelengths from the illuminated spot on the tissue;
a spectral processing classifier for determining a tissue type associated with each of a plurality of pixels of the image; and
a display device for displaying the tissue type of the plurality of pixels of the image to the surgeon.

2. The instrument of claim 1, wherein the optical system is a multiwavelength, confocal system.

3. The instrument of claim 1, further comprising apparatus for determining parameters from pixel spectra, wherein the classifier classifies each pixel according to the parameters; and wherein the parameters comprise scatter parameters of the illuminated spot corresponding to each pixel.

4. The instrument of claim 3, wherein the parameters include statistical parameters for a window comprising a plurality of pixels centered upon the pixel being classified.

5. The instrument of claim 3, wherein the parameters are corrected for absorbance of hemoglobin and deoxygenated hemoglobin in the tissue.

6. The instrument of claim 1, wherein the illuminator is a white-light illuminator.

7. The instrument of claim 1, wherein the illuminator comprises a plurality of lasers and apparatus for combining beams from the plurality of lasers.

8. The instrument of claim 7, wherein the display device is a color display device, and wherein tissue type is displayed by color coding an image of the plurality of pixels.

9. The instrument of claim 1, wherein the classifier is a K-Nearest-Neighbor classifier.

10. The instrument of claim 1, wherein the classifier is a classifier selected from the group consisting of an Artificial Neural Network classifier, a Support Vector Machine classifier, a Linear Discriminant Analysis classifier, and a Spectral Angle Mapper classifier.

11. The instrument of claim 9, wherein the classifier is trained according to normal and abnormal tissue types of a particular organ of interest.

12. The instrument of claim 1 wherein the illuminator is a supercontinuum laser.

13. The instrument of claim 1 wherein the illuminator further comprises a filter for blocking light of stimulus wavelength, and wherein the spectrally sensitive detector is capable of detecting a spectrum of the fluorescence emission.

14. The instrument of claim 13 further comprising apparatus for determining parameters from pixel spectra, wherein the parameters comprise scatter parameters of the illuminated spot corresponding to the pixel; and wherein the classifier uses measurements of light at the fluorescence wavelengths together with the parameters to classify tissue at the illuminated spot.

15. A method of performing tumor removal from tissue of an organ comprising
illuminating a surgical cavity in the tissue with a beam of light, the beam of light illuminating a spot sufficiently small on the tissue that a majority of scattered light is singly scattered;
receiving and measuring the scattered light from the tissue with a spectrally sensitive detector comprising a dispersive device and an array of photodetector elements;
adjusting measurements from the spectrally sensitive detector for hemoglobin in the tissue;
extracting scatter parameters from the measurements;
classifying tissue according to the scatter parameters, the tissue being classified as at least as tumor tissue and normal organ tissue;
displaying tissue classification information; and
removing at least some tissue classified as rapidly proliferating.

16. The method of claim 15, further comprising scanning the beam of light across the tissue, and wherein the step of displaying tissue classification information comprises constructing an image portraying a map of tissue types identified on the tissue.

17. The method of claim 16, further comprising extracting statistical parameters of a window of pixels, and wherein the step of classifying is performed according to the statistical parameters in addition to the scatter parameters.

18. The method of claim 17, wherein the beam of light is a broad spectrum light.

19. The method of claim 17, wherein the beam of light comprises composite light from a plurality of monochromatic light sources.

20. The method of claim 17 wherein the beam of light comprises light at a fluorescence stimulus wavelength, wherein the method further comprises measuring an emitted fluorescence spectrum, and wherein the step of classifying is performed according to the measured fluorescence spectrum in addition to the scatter parameters.

21. The method of claim 17, wherein the step of displaying tissue classification information comprises displaying a map of tissue classification information with rapidly proliferating tumor regions marked with a particular color different than a color marking mature tumor regions.

22. The method of claim 17, wherein the step of illuminating is performed with apparatus comprising a telecentric confocal scan lens.

23. The method of claim 17 wherein the step of classifying is performed by a K-Nearest-Neighbors type classifier that has been trained according to parameters extracted from normal and abnormal tissues of a particular organ type.

24. The method of claim 17 wherein the step of classifying is performed by an Artificial Neural Network type classifier that has been trained according to parameters extracted from normal and abnormal tissues of a particular organ type.

25. A method of mapping tissue types in an exposed organ comprising
illuminating the tissue with a beam of light, the beam of light being scanned across the tissue, the beam of light illuminating a plurality of spots sufficiently small on the tissue that a majority of scattered light is singly scattered;
for each illuminated spot on the tissue, receiving and measuring the scattered light from the tissue with a spectrophotometer;
adjusting measurements from the spectrophotometer for hemoglobin in the tissue;
extracting scatter parameters from the measurements;
classifying tissue according to the scatter parameters, the tissue being classified as at least normal organ cells and tumor cells; and
displaying tissue classification information for each spot of the plurality of spots, the classification information for each spot portrayed as a pixel of an image, the image thereby portraying a map of tissue types identified on the tissue.

26. The method of claim 25, further comprising extracting statistical parameters of a window of pixels, and wherein the step of classifying is performed according to the statistical parameters in addition to the scatter parameters.

27. The method of claim 26 wherein the statistical parameters of a window of pixels comprise textural parameters.

28. The method of claim 26, wherein the beam of light is a broad spectrum light.

29. The method of claim 26, wherein the beam of light comprises composite light from a plurality of monochromatic light sources.

30. The method of claim 26, wherein the step of illuminating is performed with apparatus comprising a confocal scan lens.

31. The method of claim 26 wherein the step of classifying is performed by a K-Nearest-Neighbors classifier that has been trained according to parameters extracted from normal and abnormal tissues of a particular organ type corresponding to the exposed organ.

32. The method of claim 26 wherein the step of classifying is performed by an Artificial Neural Network classifier that has been trained according to parameters extracted from normal and abnormal tissues of a particular organ type corresponding to the exposed organ

33. The method of claim 26 further comprising:
illuminating the tissue with a beam of light at a stimulus wavelength;
measuring a spectrum of fluorescent light from the tissue to give fluorescence data;
wherein the step of classifying is further performed using the fluorescence data.

34. The method of claim 33, wherein the step of classifying is performed using scatter parameters and fluorescence data normalized to scatter parameters measured at the stimulus wavelength.

35. The method of claim 33 further comprising measuring light from the tissue at multiple stimulus wavelengths to give multiple fluorescence data sets, and wherein the step of classifying is further performed using the multiple fluorescence data sets as well as the scatter parameters.
